(19) United States
(12) Patent Application Publication
(10) Pub. No.: US 2009/0053159 A1
(43) Pub. Date: Feb. 26, 2009

(54) Anhydrous cosmetic compositions comprising at least one silicone copolymer, at least one volatile silicone, and at least one non-volatile linear polydimethylsiloxane

(76) Inventor: Gaelle Brun, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/216,947

(22) Filed: Jul. 14, 2008

Related U.S. Application Data
(60) Provisional application No. 60/929,969, filed on Jul. 19, 2007.

(30) Foreign Application Priority Data
Jul. 13, 2007 (FR) 0756484

(51) Int. Cl. A61K 8/84; A61Q 5/12

(52) U.S. Cl. 424/70.12

(57) ABSTRACT

The present disclosure relates to anhydrous hair treatment compositions which comprise at least one copolymer based on a silicone resin and a fluid silicone, at least one linear or cyclic volatile silicone, and at least one non-volatile linear polydimethylsiloxane with a viscosity of greater than 5 cSt, wherein the at least one copolymer is present in the composition in an amount greater than 1% by weight relative to the total weight of the composition, and wherein the composition is free of pigments or comprises more than 5% by weight of at least one pigment. The present disclosure also relates to methods of treating keratin fibers using said compositions.

This application claims benefit of U.S. Provisional Application No. 60/929,969, filed Jul. 19, 2007, the contents of which are incorporated herein by reference. This application also claims benefit of priority under 35 U.S.C. § 119 to French Patent Application No. FR 0756484, filed Jul. 13, 2007, the contents of which are also incorporated herein by reference.

The present disclosure relates to compositions for treating keratin fibers, for example hair, and also to methods for treating keratin fibers comprising the use of these compositions.

Keratin fibers, for example hair, may be damaged and embrittled by the action of external environmental agents such as light and bad weather, and/or by mechanical and chemical treatments such as brushing, combing, bleaching, permanent waving, and dyeing. As a result, the keratin fibers may be difficult to manage, for example difficult to disentangle or style, and a head of hair, even a rich head of hair, may have difficulty in maintaining an attractive style due to the fact that the hair lacks vigor, volume and liveliness.

This degradation of keratin fibers may be increased by repeated permanent dyeing treatments.

To overcome this, it is possible to use styling products that allow keratin fibers to be conditioned, for example giving them body, mass, and/or volume.

These styling products may be cosmetic compositions comprising at least one polymer having a high affinity for keratin fibers. Said polymer may have the function of forming a film at the surface of said keratin fibers in order to modify their surface properties, for example to condition them.

However, the cosmetic effects imparted by use of such compositions, for example a color-effect provided by pigments, may have a tendency to dissipate, for example after a single shampoo wash.

In order to avoid this drawback, it may be envisaged to increase the persistence of the polymer deposit by performing a radical polymerization of specific monomers directly on the keratin fibers. However, such treatments may lead to degradation of the keratin fibers and hair thus treated may be difficult to disentangle.

It is also known to coat keratin fibers with a composition comprising at least one electrophilic monomer of cyanoacrylate type, for example as described in patent application FR 2 833 489. Such a treatment may result in perfectly coated and non-greasy hair. However, the coating obtained requires particular operating conditions due to the reactivity of the electrophilic monomer. Moreover, the coating obtained with these electrophilic monomers may become tacky with fatty substances such as sebum.

There exist silicone copolymers comprising a silicone resin portion and a fluid silicone portion, for example the silicone copolymers known as Bio-PSA®. These copolymers are described, as a nonlimiting example, in PCT publications WO 03/026 596, WO 2004/073 626, WO 2007/051 505, and WO 2007/051 506 for various cosmetic applications, such as application to the hair, the nails, and the skin.

It is desirable to develop easy-to-use methods for treating keratin fibers, for example hair, which can produce coatings that are resistant with respect to shampooing and to the various environmental agents and mechanical and chemical treatments to which the hair may be exposed, for example blow-drying and perspiration, while at the same time showing tolerance to fatty substances such as sebum. It is also desirable to obtain easy-to-use colored coatings, which are resistant to environmental agents and mechanical and chemical treatments and which do not degrade the integrity of keratin fibers.

Thus, disclosed herein are anhydrous keratin fiber treatment compositions comprising at least one copolymer based on a silicone resin and a fluid silicone, at least one linear or cyclic volatile silicone, and at least one non-volatile linear polydimethylsiloxane with a viscosity of greater than 5 cSt, wherein the at least one copolymer is present in the composition in an amount greater than 1% by weight relative to the total weight of the composition, and wherein the composition does not comprise coloring pigments.

Also disclosed herein are anhydrous compositions for dyeing keratin fibers, comprising at least one copolymer based on a silicone resin and a fluid silicone, at least one linear or cyclic volatile silicone, at least one non-volatile linear polydimethylsiloxane with a viscosity of greater than 5 cSt, and at least one coloring pigment, wherein the at least one copolymer is present in the composition in an amount greater than 1% by weight relative to the total weight of the composition, and coloring pigments are present in the composition in a total amount greater than 5% by weight relative to the total weight of the composition.

Also disclosed herein are methods of treating keratin fibers, which make it possible to obtain an effect that is resistant with respect to shampooing, wherein the methods comprise the use of at least one composition according to the present disclosure.

Also disclosed herein is the use of anhydrous compositions comprising at least one copolymer based on a silicone resin and a fluid silicone with at least one coloring pigment to obtain a colored coating, or in the absence of coloring pigments to obtain an uncolored coating.

In one embodiment of the present disclosure, coatings may be obtained on the keratin fibers that give the fibers shampoo-resistant volume, mass, and body, while at the same time maintaining the physical qualities of the keratin fiber. In one aspect of the present disclosure, when a composition comprising at least one coloring pigment is used, coatings may be obtained on the keratin fibers that give the fibers a long-lasting coloring effect. Such coatings may also be resistant to the external factors to which the fibers may be exposed, such as blow-drying or perspiration. In one embodiment of the present disclosure, it is possible to obtain permanent coloring effects without using oxidizing agents liable to degrade keratin fibers.

Coatings obtained according to the present disclosure may be in the form of a smooth, uniform deposit and may have excellent adhesion to keratin fibers. Moreover, it has been found, surprisingly, that keratin fibers treated according to the present disclosure may remain individualized and may be easily styled, and that the styling properties thus afforded to the keratin fibers may be shampoo-resistant.

### Copolymer Based on Silicone Resin and on Fluid Silicone

Disclosed herein are silicone copolymers derived from the reaction between a silicone resin and a fluid silicone.

Non-limiting examples of such copolymers are described in “Silicone Pressure Sensitive Adhesive”, Sobieski and Tangney, Handbook of Pressure Sensitive Adhesive Technology (D. Satas Ed.), Von Nostrand Reinhold, New York.

In the at least one silicone copolymer according to the present disclosure, the silicone resin may be present in an amount ranging from 45% to 75% (relative to the total mass of silicone) and the fluid silicone may present in an amount ranging from 25% to 55%, the sum of the percentage amounts of silicone resin and of fluid silicone being equal to 100. For example, the silicone resin may be present in an amount ranging from 55% to 65% (relative to the total mass of silicone) and the fluid silicone may present in an amount ranging from 35% to 45%, the sum of the percentage amounts of silicone resin and of fluid silicone being equal to 100.

In at least one embodiment of the present disclosure, the silicone resin is the condensation product of SiO₂ groups and of R₃(SiO)_{1/2} (triorganosilyl) groups, wherein for each group, R is independently chosen from methyl, ethyl, propyl, and vinyl radicals, and wherein the ratio between the SiO₂ functions and the R₃(SiO)_{1/2} functions within the resulting silicone resin ranges from 0.6 to 0.9. Non-limiting examples of triorganosilyl groups that may be used to form the silicone resin include, trimethylsilyl, triethylsilyl, methylmethylpropylsilyl, dimethylvinylsilyl groups, and mixtures thereof. In one embodiment of the present disclosure, the triorganosilyl groups are trimethylsilyl groups.

In at least one embodiment of the present disclosure, the fluid silicone is a diorganopolysiloxane bearing —OH end functions and having a viscosity ranging from 100 to 100,000 cSt at 25° C., wherein the substituents of the diorganopolysiloxane are independently chosen from methyl, ethyl, propyl, and vinyl radicals. For example, the diorganopolysiloxane may be chosen from, in a non-limiting manner, a polydimethylsiloxane, an ethylmethyl polysiloxane, a copolymer of dimethylsiloxane and of methylvinylsiloxane, and mixtures of such polymers and copolymers bearing —OH end functions. In one embodiment, the diorganopolysiloxanes may be linear. In one embodiment, the diorganopolysiloxane is a polydimethylsiloxane.

Syntheses of silicone copolymers such as those according to the present disclosure are described in, for example, but not limited to, U.S. Pat. No. 5,162,410 or in Canadian patent CA 711 756.

In one embodiment of the present disclosure, silicone copolymers according to the present disclosure may be prepared, for example, by heating the following mixture:

1. from 45% to 75% by mass of silicone resin, said resin being the condensation product of SiO₂ and R₃(SiO)_{1/2} groups wherein for each group R is independently selected from methyl, ethyl, propyl, and vinyl radicals, and wherein the ratio between the SiO₂ functions and the R₃(SiO)_{1/2} functions within said silicone resin ranges from 0.6 to 0.9;

2. from 25% to 55% by mass of fluid diorganopolysiloxane bearing —OH end functions and having a viscosity ranging from 100 to 100,000 cSt at 25° C., wherein the substituents of the diorganopolysiloxane are independently chosen from methyl, ethyl, propyl, and vinyl radicals;

3. from 0.001% to 5% of a suitable catalyst, such as an organic aliphatic amine compound, for example chosen from primary amines, secondary amines, tertiary amines, carboxylic acid salts of the amines mentioned above, and quaternary ammonium salts.

The mixture is heated to a temperature ranging from 80° C. to 160° C. until the adhesive nature of the resulting silicone copolymer is obtained.

As silicone copolymers according to the present disclosure, non-limiting mention may be made of copolymers sold by Dow Corning under the reference Bio-PSA®. These Bio-PSA® copolymers may be in two forms, standard or amine-compatible, and may be provided in different solvents and with different ratios of silicone resin and fluid silicone. For example, non-limiting mention may be made of the Bio-PSA® grades 7-4400, 7-4500, and 7-4600. In at least one embodiment of the present disclosure, the silicone copolymer is Bio-PSA® grade 7-4400.

The at least one silicone copolymer may be present in compositions according to the present disclosure in an amount ranging from greater than 1% to 40% by weight, for example ranging from 1.5% to 20% by weight, for further example ranging from 1.5% to 15% by weight, relative to the total weight of the composition.

### Volatile Silicone

As used herein, the term “volatile silicone” is understood to mean a silicone that is liquid at room temperature (25° C.) and at atmospheric pressure, with a vapor pressure at 25° C. of greater than 0.1 mmHg, for example ranging from 0.1 to 300 mmHg, for example ranging from 0.1 to 200 mmHg.

As volatile silicones, non-limiting examples include linear and cyclic silicones comprising from 4 to 7 silicon atoms, these silicones optionally further comprising C₁-C₁₀ alkyl and/or C₁-C₁₀ alkoxy groups. For example, volatile silicones that may be used in compositions according to the present disclosure may be chosen from octamethylcyclotetrasiloxane, decamethylcyclopentasiloxane, dodecamethylcyclohexasiloxane, heptamethylhexyltrisiloxane, heptamethylethyltrisiloxane, heptamethyloctyltrisiloxane, octamethyltrisiloxane, decamethyltetrasiloxane, and mixtures thereof.

In one embodiment of the present disclosure, the volatile silicone is cyclic and may be chosen from decamethylcyclopentasiloxane, octamethyltrisiloxane, and decamethyltetrasiloxane.

Additional non-limiting examples of volatile silicones that may be used in compositions according to the present disclosure include the decamethylcyclopentasiloxane sold under the name DC-245 by the company Dow Corning, the octamethyltrisiloxane sold under the name DC-200 Fluid 1 cSt by the company Dow Corning, and the decamethyltetrasiloxane sold under the name DC-200 Fluid 1.5 cSt by the company Dow Corning.

In at least one embodiment of the present disclosure, the volatile silicone is a cyclic volatile silicone having a low viscosity, for example a viscosity of less than 5 cSt at 25° C.

In at least one embodiment of the present disclosure, the volatile silicone is the decamethylcyclopentasiloxane sold under the name DC-245 by the company Dow Corning.

The at least one volatile silicone may be present in compositions according to the present disclosure in an amount ranging from 0.1% to 99% by weight, for example ranging from 1% to 95% by weight, for further example ranging from 5% to 90% by weight, relative to the total weight of the composition.

Non-Volatile Linear PDMS with a Viscosity of Greater than 5 cSt

Non-volatile linear polydimethylsiloxanes (PDMS) with a viscosity of greater than 5 cSt that may be used in compositions according to the present disclosure may be chosen from, but are not limited to, silicone gums and silicone oils with a vapor pressure of less than 0.1 mmHg at 25° C.

As non-limiting examples, the non-volatile linear PDMS with a viscosity of greater than 5 cSt may be chosen from polydimethylsiloxanes; alkyl dimethicones; polyphenylmethylsiloxanes, such as phenyl dimethicones, phenyl trimethicones and vinyl methyl methicones; and silicones modified with optionally fluorinated aliphatic and/or aromatic groups, and/or with functional groups such as hydroxyl, thiol, and/or amine groups.

In one embodiment of the present disclosure, the viscosity of the non-volatile linear PDMS may be greater than 5 cSt at 25° C. For example, the viscosity of the non-volatile linear PDMS may range from 5 to 5,000,000 cSt, for example from 100 to 4,000,000 cSt, for further example from 5,000 to 4,000,000 cSt.

In one embodiment of the present disclosure, the non-volatile linear PDMS may have a molecular weight ranging from 500 to 800,000 g/mol, for example from 5,000 to 700,000 g/mol, and for further example from 50,000 to 600,000 g/mol.

As non-volatile linear polydimethylsiloxanes that may be used in compositions according to the present disclosure, non-limiting mention may be made of silicones of formula (II):

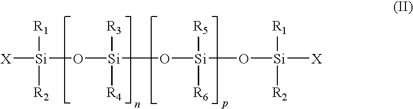

wherein:

R₁, R₂, R₅ and R₆ are, independently of one another, chosen from C₁-C₆ alkyl radicals,

R₃ and R₄ are, independently of one another, chosen from C₁-C₆ alkyl radicals, vinyl radicals, and aryl radicals,

X is chosen from C₁-C₆ alkyl radicals, hydroxyl radicals, vinyl radicals, and amine radicals,

n and p are integers chosen so as to have a compound with a viscosity of greater than 5 cSt; for example, the sum (n+p) may be greater than 10.

As non-volatile linear polydimethylsiloxanes that may be used in compositions according to the present disclosure, non-limiting examples include:

silicones of formula (II) wherein R₁, R₂, R₃, R₄, R₅, R₆, and X are methyl radicals, for example the product sold under the name Baysilicone TP 3898 by the company General Electric, and the product sold under the name AK 500 000 by the company Wacker;

silicones of formula (II) wherein R₁, R₂, R₃, R₄, R₅, R₆, and X are methyl radicals, and p and n are chosen such that the molecular weight is about 120,000 g/mol, for example the product sold under the name Dow Corning 200 Fluid 60 000 CS by the company Dow Corning;

silicones of formula (II) wherein R₁, R₂, R₃, R₄, R₅, R₆, and X are methyl radicals, and p and n are chosen such that the molecular weight is about 250,000 g/mol, for example the product sold under the name Mirasil DM 500 000 by the company Rhodia and the product sold under the name Dow Corning 200 Fluid 500 000 cSt by the company Dow Corning;

silicones of formula (II) wherein R₁, R₂, R₃, R₄, R₅, and R₆ are methyl radicals, X is a hydroxyl radical, and n and p are chosen such that the molecular weight of the polymer is about 600,000 g/mol, for example the product sold under the name SGM 36 by the company Dow Corning; and
dimethicones of the (polydimethylsiloxane)(methylvinylsiloxane) type, for example the product sold under the name SE63 by GE Bayer Silicones, and poly(dimethylsiloxane)(diphenyl) (methylvinylsiloxane) copolymers, and mixtures thereof.

In one embodiment of the present disclosure, the non-volatile linear polydimethylsiloxanes are oxyalkylenated.

### Pigments

In one embodiment of the present disclosure, the composition is a keratin fiber dyeing composition comprising at least one coloring pigment. Such a composition may make it possible to obtain long-lasting colored coatings, without degrading the keratin fibers.

As used herein, the term “coloring pigments” is understood to mean any pigment that gives color to keratin materials. This is understood to exclude white pigments, such as titanium dioxide, which only make keratin materials white.

Coloring pigments that may be used in compositions according to the present disclosure may be chosen from organic and/or mineral pigments known in the art, for example, but not limited to, those described in Kirk-Othmer's Encyclopaedia of Chemical Technology and in Ullmann's Encyclopaedia of Industrial Chemistry.

The at least one coloring pigment may be in the form of a pigmentary powder or a paste. It may be coated or uncoated.

The coloring pigment may be chosen, for example, from mineral pigments, organic pigments, lakes, pigments with special effects such as nacres or glitter flakes, and mixtures thereof.

In one aspect of the present disclosure, the at least one coloring pigment may be chosen from mineral pigments. As used herein, the term “mineral pigment” is understood to mean any pigment that satisfies the definition provided in Ullmann's Encyclopaedia in the chapter on inorganic pigments. Non-limiting examples of mineral pigments include: iron oxides, chromium oxides, manganese violet, ultramarine blue, chromium hydrate, and ferric blue.

In one aspect of the present disclosure, the coloring pigment may be chosen from organic pigments. As used herein, the term “organic pigment” is understood to mean any pigment that satisfies the definition provided in Ullmann's Encyclopaedia in the chapter on organic pigments. Non-limiting examples of organic pigments include: nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane, and quinophthalone compounds.

As non-limiting examples, organic pigments may be chosen from carmine, carbon black, aniline black, melanin, azo yellow, quinacridone, phthalocyanin blue, sorghum red, the blue pigments codified in the Color Index under the references CI 42090, 69800, 69825, 73000, 74100, and 74160, the yellow pigments codified in the Color Index under the references CI 11680, 11710, 15985, 19140, 20040, 21100, 21108, 47000, and 47005, the green pigments codified in the Color Index under the references CI 61565, 61570, and 74260, the orange pigments codified in the Color Index under the references CI 11725, 15510, 45370, and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, and 75470, and pigments obtained by oxidative polymerization of indole or phenolic derivatives, for example, as described in patent FR 2 679 771.

As pigmentary pastes comprising organic pigments, non-limiting examples include the products sold by the company Hoechst under the names:

Jaune Cosmenyl IOG: Pigment Yellow 3 (CI 11710);

Jaune Cosmenyl G: Pigment Yellow 1 (CI 11680);

Orange Cosmenyl GR: Pigment Orange 43 (CI 71105);

Rouge Cosmenyl R: Pigment Red 4 (CI 12085);

Carmine Cosmenyl FB: Pigment Red 5 (CI 12490);

Violet Cosmenyl RL: Pigment Violet 23 (CI 51319);

Bleu Cosmenyl A2R: Pigment Blue 15.1 (CI 74160);

Vert Cosmenyl GG: Pigment Green 7 (CI 74260); and

Noir Cosmenyl R: Pigment Black 7 (CI 77266).

In one embodiment, the at least one coloring pigment that may be used in compositions according to the present disclosure may be in the form of composite pigments, for example as described in patent EP 1 184 426. For example, these composite pigments may be, but are not limited to, compounds of particles comprising a mineral core, at least one binder for ensuring the binding of the organic pigments to the core, and at least one organic pigment at least partially covering the core.

In one embodiment of the present disclosure, the organic pigment may be a lake. As used herein, the term “lake” is understood to mean insolubilized dyes adsorbed onto insoluble particles, the assembly thus obtained remaining insoluble during use.

The inorganic substrates onto which the dyes are adsorbed may be chosen, for example, from alumina, silica, calcium sodium borosilicate, calcium aluminum borosilicate, and aluminum.

Non-limiting examples of dyes include cochineal carmine and the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), and D&C Blue 1 (CI 42 090).

As a lake, non-limiting mention may be made of the product known under the following name: D&C Red 7 (CI 15 850:1).

In one aspect of the present disclosure, the at least one coloring pigment may be chosen from pigments with special effects. As used herein, the term “pigments with special effects” is understood to mean pigments that create a non-uniform colored appearance (for example, characterized by a certain shade, a certain vivacity, and/or a certain lightness) that changes as a function of the conditions of observation (for example, light, temperature, observation angles, etc.). Pigments with special effects may thus contrast with white or colored pigments that create a standard uniformly opaque, semi-transparent, or transparent colored appearance.

Several types of pigments with special effects exist: for example, but not limited to, those with a low refractive index, such as fluorescent, photochromic, and thermochromic pigments, and those with a high refractive index, such as nacres and glitter flakes.

As pigments with special effects, non-limiting examples include: nacreous pigments, for example white nacreous pigments such as mica coated with titanium or with bismuth oxychloride, colored nacreous pigments such as titanium mica with iron oxides, titanium mica with ferric blue, titanium mica with chromium oxide, titanium mica with an organic pigment such as the above-mentioned type, and nacreous pigments based on bismuth oxychloride. As nacreous pigments, non-limiting examples include: Cellini nacres sold by Engelhard (mica-TiO₂-lake), Prestige sold by Eckart (mica-TiO₂), Prestige Bronze sold by Eckart (mica-Fe₂O₃), and Colorona sold by Merck (mica-TiO₂—Fe₂O₃).

In addition to nacres on a mica support, multilayer pigments based on synthetic substrates such as alumina, silica, sodium calcium borosilicate, calcium aluminum borosilicate, and aluminum, may be envisaged within the scope of the present disclosure.

In one aspect of the present disclosure, the at least one coloring pigment may be chosen from pigments with an interference effect that are not fixed onto a substrate, for example liquid crystals (for example, Helicones HC from Wacker), holographic interference flakes (for example, Geometric Pigments and Spectra f/x from Spectratek). Pigments with special effects also comprise fluorescent pigments, for example pigments that are fluorescent in daylight and pigments that produce an ultraviolet fluorescence, phosphorescent pigments, photochromic pigments, thermochromic pigments, and quantum dots, for example the products sold by the company Quantum Dots Corporation.

Quantum dots are luminescent semiconductive nanoparticles capable of emitting, under light excitation, radiation with a wavelength of between 400 nm and 700 nm. These nanoparticles are known from the literature. They may be manufactured, for example, as described in U.S. Pat. Nos. 6,225,198 and 5,990,479, in the publications cited therein, and in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997, pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029.

The variety of pigments that may be used in compositions according to the present disclosure makes it possible to obtain a wide range of colors, and also optical effects, for example metallic effects and/or interference effects.

In one embodiment, the at least one coloring pigments used in cosmetic compositions according to the present disclosure may range in size from 10 nm to 200 μm, for example from 20 nm to 80 μm, and for example from 30 nm to 50 μm.

In one embodiment of the present disclosure, the at least one coloring pigment used in cosmetic compositions according to the present disclosure may be dispersed in the composition by means of a dispersant.

As used herein, the term “dispersant” is understood to mean a compound that serves to protect dispersed particles against agglomeration or flocculation. Dispersants may be chosen from surfactants, oligomers, polymers, and mixtures thereof, bearing one or more functionalities with strong affinity for the surface of the particles to be dispersed. For example, dispersants may physically or chemically attach to the surface of pigments. Dispersants may also contain at least one functional group that is compatible with or soluble in the continuous medium. Non-limiting examples of dispersants that may be used in compositions according to the present disclosure include 12-hydroxystearic acid esters and C₈ to C₂₀ fatty acid esters of polyols such as glycerol and diglycerol, for example poly(12-hydroxystearic acid) stearates with a molecular weight of about 750 g/mol, for example the product sold under the name Solsperse 21000 by the company Avecia and polyglyceryl-2 dipolyhydroxystearate (CTFA name) sold under the reference Dehymyls PGPH by the company Henkel, and polyhydroxystearic acids, for example the product sold under the reference Arlacel P100 by the company Uniqema, and mixtures thereof.

Further non-limiting examples of dispersants that may be used in compositions according to the present disclosure include quaternary ammonium derivatives of polycondensed fatty acids, for example Solsperse 17000 sold by the company Avecia, and polydimethylsiloxane/oxypropylene mixtures, for example those sold by the company Dow Corning under the references DC2-5185 and DC2-5225 C.

In one aspect of the present disclosure, the at least one coloring pigment used in cosmetic compositions according to the present disclosure may be surface-treated with an organic agent.

As pigments that have been surface-treated which may be dispersed in compositions according to the present disclosure, non-limiting examples include pigments that have undergone a total or partial surface treatment of chemical, electronic, electrochemical, mechanochemical and/or mechanical nature, with an organic agent, for example those described in Cosmetics and Toiletries, February 1990, Vol. 105, pp. 53-64. The said organic agent may be chosen, for example, from amino acids; waxes, for example carnauba wax and beeswax; fatty acids, fatty alcohols, and derivatives thereof, for example stearic acid, hydroxystearic acid, stearyl alcohol, hydroxystearyl alcohol, lauric acid, and derivatives thereof; anionic surfactants; lecithins; sodium, potassium, magnesium, iron, titanium, zinc, and aluminum salts of fatty acids, for example aluminum stearate and aluminum laurate; metal alkoxides; polysaccharides, for example chitosan, cellulose and derivatives thereof; polyethylene; (meth)acrylic polymers, for example polymethyl methacrylates; polymers and copolymers containing acrylate units; proteins; alkanolamines; silicone compounds, for example silicones, polydimethylsiloxanes, alkoxysilanes, alkylsilanes, and siloxysilicates; organofluorine compounds, for example perfluoroalkyl ethers; and fluorosilicone compounds.

The surface-treated pigments which may be dispersed in compositions according to the present disclosure may also have been treated with a mixture of the above-mentioned organic agents and/or may have undergone several surface treatments.

The surface-treated pigments which may be dispersed in compositions according to the present disclosure may be prepared according to surface-treatment techniques known to those skilled in the art, and/or may be commercially available in the required form.

In at least one aspect, the surface-treated pigments which may be dispersed in compositions according to the present disclosure are coated with an organic layer.

The organic agent with which the pigments are treated may be deposited on the pigments by evaporation of solvent, chemical reaction between the molecules of the surface agent, and/or creation of a covalent bond between the surface agent and the pigments.

The surface treatment may thus be performed, for example, by chemical reaction of a surface agent with the surface of the pigments and creation of a covalent bond between the surface agent and the pigments or the fillers. Such a method is described, for example, in U.S. Pat. No. 4,578,266.

In at least one embodiment of the present disclosure, an organic agent covalently bonded to the at least one coloring pigment may be used.

The surface treatment agent may be present in an amount ranging from 0.1% to 50% by weight, for example from 0.5% to 30% by weight, for example from 1% to 10% by weight, relative to the total weight of the surface-treated pigments.

In at least one aspect of the present disclosure, the surface treatments of the at least one coloring pigment are chosen from the following treatments:

a PEG-silicone treatment, for example the AQ surface treatment sold by LCW;

a chitosan treatment, for example the CTS surface treatment sold by LCW;

a triethoxycaprylylsilane treatment, for example the AS surface treatment sold by LCW;

a methicone treatment, for example the SI surface treatment sold by LCW;

a dimethicone treatment, for example the Covasil 3.05 surface treatment sold by LCW;

a dimethicone/trimethyl siloxysilicate treatment, for example the Covasil 4.05 surface treatment sold by LCW;

a lauroyllysine treatment, for example the LL surface treatment sold by LCW;

a lauroyllysine dimethicone treatment, for example the LL/SI surface treatment sold by LCW;

a magnesium myristate treatment, for example the MM surface treatment sold by LCW;

an aluminum dimyristate treatment, for example the Ml surface treatment sold by Miyoshi;

a perfluoropolymethylisopropyl ether treatment, for example the FHC surface treatment sold by LCW;

an isostearyl sebacate treatment, for example the HS surface treatment sold by Miyoshi;

a disodium stearoyl glutamate treatment, for example the NAI surface treatment sold by Miyoshi;

a dimethicone/disodium stearoyl glutamate treatment, for example the SA/NAI surface treatment sold by Miyoshi;

a perfluoroalkyl phosphate treatment, for example the PF surface treatment sold by Daito;

an acrylate/dimethicone copolymer and perfluoroalkyl phosphate treatment, for example the FSA treatment sold by Daito;

a polymethylhydrogenosiloxane/perfluoroalkyl phosphate treatment, for example the FS01 surface treatment sold by Daito;

a lauroyllysine/aluminum tristearate treatment, for instance the LL-AlSt surface treatment sold by Daito;

an octyltriethylsilane treatment, for example the OTS surface treatment sold by Daito;

an octyltriethylsilane/perfluoroalkyl phosphate treatment, for example the FOTS surface treatment sold by Daito;

an acrylate/dimethicone copolymer treatment, for example the ASC surface treatment sold by Daito;

an isopropyl titanium triisostearate treatment, for example the ITT surface treatment sold by Daito;

a microcrystalline cellulose and carboxymethylcellulose treatment, for example the AC surface treatment sold by Daito;

a cellulose treatment, for example the C2 surface treatment sold by Daito;

an acrylate copolymer treatment, for example the APD surface treatment sold by Daito; and

a perfluoroalkyl phosphate/isopropyl titanium triisostearate treatment, for example the PF+ITT surface treatment sold by Daito.

Compositions according to the present disclosure may further comprise at least one coloring pigment that has not been surface treated.

In one embodiment, coloring pigments that have not been surface treated may be present in compositions according to the present disclosure in a total amount ranging up to 40%, for example ranging up to 20%, by weight relative to the total weight of the composition.

Compositions according to the present disclosure may further comprise other colored and/or coloring species, for example hydrophilic, hydrophobic, and amphiphilic direct dyes and dye precursors.

Compositions according to the present disclosure may further comprise other constituents. In one embodiment, compositions according to the present disclosure may comprise a non-silicone organic solvent, which may be volatile or non-volatile. The term “volatile” is understood to have the same meaning as that previously discussed.

Non-limiting examples of volatile organic solvents include:

volatile C₁-C₄ alkanols, for example ethanol and isopropanol;

volatile C₅-C₇ alkanes, for example n-pentane, hexane, cyclopentane, 2,3-dimethylbutane, 2,2-dimethylbutane, 2-methylpentane, and 3-methylpentane;

esters of liquid C₁-C₂₀ acids and esters of volatile C₁-C₈ alcohols, for example methyl acetate, n-butyl acetate, ethyl acetate, propyl acetate, isopentyl acetate, and ethyl 3-ethoxypropionate;

ketones that are liquid at room temperature and volatile, for example methyl ethyl ketone, methyl isobutyl ketone, diisobutyl ketone, isophorone, cyclohexanone, and acetone;

volatile polyols, for example propylene glycol;

volatile ethers, for example dimethoxymethane, diethoxyethane, and diethyl ether;

volatile glycol ethers, for example 2-butoxyethanol, butyl diglycol, diethylene glycol monomethyl ether, propylene glycol n-butyl ether, and propylene glycol monomethyl ether acetate;

volatile hydrocarbon-based oils, for example volatile hydrocarbon-based oils comprising from 8 to 16 carbon atoms, and mixtures thereof, for example branched C₈-C₁₆ alkanes, for example C₈-C₁₆ isoalkanes (which may be known as isoparaffins), isododecane, isodecane, and, for example, the oils sold under the trade names Isopar or Permethyl, and mixtures thereof. As volatile hydrocarbon-based oils, non-limiting mention may also be made of isohexyl neopentanoate and isodecyl neopentanoate;

volatile C₄-C₁₀ perfluoroalkanes, for example dodecafluoropentane, tetradecafluorohexane, and decafluoropentane;

volatile perfluorocycloalkyls, for example perfluoromethylcyclopentane, 1,3-perfluorodimethylcyclohexane, and perfluorodecalin, for example the products sold, respectively, under the names Flutec PC1®, Flutec PC3®, and Flutec PC6® by the company F2 Chemicals, and perfluorodimethylcyclobutane and perfluoromorpholine; and

volatile fluoroalkyl or heterofluoroalkyl compounds of the following formula:

CH₃—(CH₂)ₙ-[Z]ₜ-X—CF₃

wherein t is 0 or 1; n is 0, 1, 2 or 3; X is chosen from linear and branched divalent C₂-C₅ perfluoroalkyl radicals, and Z is chosen from O, S, and NR, wherein R is chosen from hydrogen atoms and —(CH₂)ₙ—CH₃ and —(CF₂)ₘ—CF₃ radicals, wherein m is 2, 3, 4 or 5.

Non-limiting examples of volatile fluoroalkyl and heterofluoroalkyl compounds include methoxynonafluorobutane, for example the products sold under the name MSX 4518® and HFE-7100® by the company 3M, and ethoxynonafluorobutane, for example the product sold under the name HFE-7200® by the company 3M.

Non-limiting examples of non-volatile organic solvents include:

non-volatile aromatic alcohols, for example benzyl alcohol and phenoxyethanol;

esters of liquid C₁-C₂₀ acids and esters of non-volatile C₁-C₈ alcohols, for example isopropyl myristate;

ethylene carbonate, propylene carbonate, and butylene carbonate;

non-volatile polyols, for example glycerol, ethylene glycol, dipropylene glycol, and butylene glycol;

non-volatile glycol ethers, for example diethylene glycol monomethyl ether and dipropylene glycol mono-n-butyl ether;

non-volatile hydrocarbon-based oils, for example isohexadecane;

non-volatile liquid C₁₀-C₃₀ alcohols, for example oleyl alcohol; esters of liquid C₁₀-C₃₀ fatty alcohols, for example benzoates of C₁₀-C₃₀ fatty alcohols and mixtures thereof; polybutene oil, isononyl isononanoate, isostearyl malate, pentaerythrityl tetraisostearate, and tridecyl trimellitate; and

non-volatile perfluoro solvents, for example perfluoroperhydrophenanthrene, for example the product sold under the name Flutec PC11® by the company F2 Chemicals.

In at least one embodiment, compositions according to the present disclosure may comprise an organic solvent chosen such that its boiling point is less than 200° C.

In at least one embodiment, compositions according to the present disclosure may comprise a volatile organic solvent chosen, for example, from ethanol, isopropanol, acetone, and isododecane.

An organic solvent may be present in compositions according to the present disclosure in an amount ranging from 0.1% to 90%, for example from 1% to 80%, for example from 5% to 70%, by weight, relative to the total weight of the composition.

Compositions according to the present disclosure may further comprise additional silicone compounds other than those described previously.

For example, compositions according to the present disclosure may contain a grafted silicone polymer. For the purposes of the present disclosure, the term “grafted silicone polymer” is understood to mean a polymer comprising a polysiloxane portion and a non-silicone organic portion, one of the two portions constituting the main chain of the polymer and the other being grafted onto the said main chain.

Grafted silicone polymers which may be used in cosmetic compositions according to the present disclosure may be chosen from, but are not limited to, polymers with a non-silicone organic backbone (main chain) grafted with monomers containing a polysiloxane, polymers with a polysiloxane backbone (main chain) grafted with non-silicone organic monomers, and mixtures thereof.

The non-silicone organic monomers constituting the main chain of the grafted silicone polymer may be chosen from, but are not limited to, radical-polymerizable ethylenically unsaturated monomers, polycondensation-polymerizable monomers, for example those forming polyamides, polyesters, polyurethanes, and ring-opening-polymerizable monomers, for example oxazoline and caprolactone type monomers.

In one embodiment of the present disclosure, the polymers comprising a non-silicone organic backbone (main chain) grafted with monomers containing a polysiloxane may be chosen from those described in U.S. Pat. Nos. 4,693,935, 4,728,571, and 4,972,037, and patent applications EP-A-0 412 704, EP-A-0 412 707, EP-A-0 640 105, and WO 95/00578. These copolymers may be obtained, for example, by free-radical polymerization starting with ethylenically unsaturated monomers and monomers having a terminal vinyl group, or alternatively by reaction of a polyolefin comprising functionalized groups and a polysiloxane macromer having a terminal function which is reactive with the said functionalized groups.

In one aspect of the present disclosure, the grafted silicone polymers comprise:

a) from 0 to 98% by weight of at least one radical-polymerizable ethylenically unsaturated lipophilic monomer (A) of low lipophilic polarity;

b) from 0 to 98% by weight of at least one ethylenically unsaturated polar hydrophilic monomer (B), which is copolymerizable with the monomer(s) of the type (A);

c) from 0.01% to 50% by weight of at least one polysiloxane macromer (C) of general formula (VI):

X—(Y)ₙ—Si(R₍₃₋ₘ₎Zₘ) (VI)

wherein:

X is a vinyl group which is copolymerizable with the monomers (A) and (B);

Y is a divalent bonding group;

R is chosen from hydrogen atoms and, C₁-C₆ alkyl, C₁-C₆ alkoxy, and C₆-C₁₂ aryl radicals;

Z is a monovalent polysiloxane unit with a number-average molecular weight of at least 500 g/mol;

n is 0 or 1 and m is 1, 2, or 3;

the percentages by weight being calculated relative to the total weight of the monomers (A), (B), and (C).

In one embodiment, the grafted silicone polymers described above have a number-average molecular weight ranging from 10,000 to 2,000,000 g/mol. In one embodiment, the grafted silicone polymers described above have a glass transition temperature (T_{g}) or a crystal melting temperature (Tₘ) of at least −20° C.

Non-limiting examples of lipophilic monomers (A) include: acrylic and methacrylic acid esters of C₁-C₂₄ alcohols; styrene; polystyrene macromers; vinyl acetate; vinyl propionate; α-methylstyrene; tert-butylstyrene; butadiene; cyclohexadiene; ethylene; propylene; vinyltoluene; acrylic and methacrylic acid esters of 1,1-dihydroperfluoroalkanols and of derivatives thereof; acrylic and methacrylic acid esters of ω-hydrofluoroalkanols; acrylic and methacrylic acid esters of fluoroalkylsulfonamido alcohols; acrylic and methacrylic acid esters of fluoroalkyl alcohols; acrylic and methacrylic acid esters of fluoroether alcohols; and mixtures thereof. In at least one embodiment, lipophilic monomers (A) are chosen from n-butyl methacrylate, isobutyl methacrylate, tert-butyl acrylate, tert-butyl methacrylate, 2-ethylhexyl methacrylate, methyl methacrylate, 2-(N-methylperfluorooctanesulfonamido)ethyl acrylate, 2-(N-butylperfluorooctanesulfonamido)ethyl acrylate, heptadecafluorooctylmethylaminoethyl methacrylate, and mixtures thereof.

Non-limiting examples of polar monomers (B) include: acrylic acid, methacrylic acid, N,N-dimethylacrylamide, dimethylaminoethyl methacrylate, quaternized dimethylaminoethyl methacrylate, (meth)acrylamide, N-t-butylacrylamide, maleic acid, maleic anhydride and hemiesters thereof, hydroxyalkyl (meth)acrylates, diallyldimethylammonium chloride, vinylpyrrolidone, vinyl ethers, maleimides, vinylpyridine, vinylimidazole, heterocyclic vinyl polar compounds, styrene sulfonate, allyl alcohol, vinyl alcohol, vinylcaprolactam, and mixtures thereof. In at least one embodiment, polar monomers (B) are chosen from acrylic acid, N,N-dimethylacrylamide, dimethylaminoethyl methacrylate, quaternized dimethylaminoethyl methacrylate, vinylpyrrolidone, and mixtures thereof.

In one aspect of the present disclosure, the polysiloxane macromers (C) of formula (VI) may be chosen from compounds of formula (VII) below:

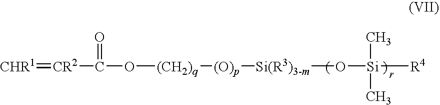

wherein:

R¹ is chosen from hydrogen atoms and —COOH radicals;

R² is chosen from hydrogen atoms and methyl and —CH₂COOH radicals;

R³ is chosen from C₁-C₆ alkyl, alkoxy, and alkylamino radicals, C₆-C₁₂ aryl radicals, and hydroxyl radicals; for example, R³ may be a methyl radical;

R⁴ is chosen from C₁-C₆ alkyl, alkoxy and alkylamino radicals, C₆-C₁₂ aryl radicals, and hydroxyl radicals; for example, R⁴ may be a methyl radical;

q is an integer ranging from 2 to 6; for example, q may be 3;

p is 0 or 1, for example, p may be 0;

r is an integer ranging from 5 to 700; and

m is an integer ranging from 1 to 3; for example, m may be 1.

Non-limiting examples of polysiloxane macromers (C) include those of formula:

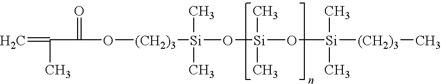

wherein n is an integer ranging from 5 to 700.

In one embodiment of the present disclosure, the polymers comprising a non-silicone organic backbone (main chain) grafted with monomers containing a polysiloxane may have the following structure:

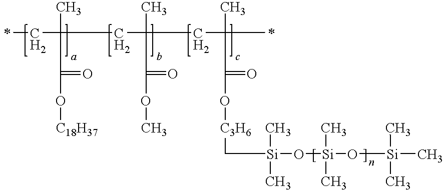

One example of such a polymer is the product sold under the name KP 561 by the company Shin-Etsu.

In another embodiment of the present disclosure, the polymer with a non-silicone organic backbone (main chain) grafted with monomers containing a polysiloxane may have the following structure:

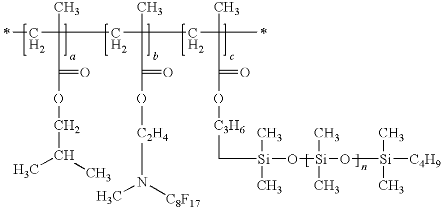

One example of such a polymer is Polysilicone 7, sold under the name SA70 by 3M.

Other non-limiting examples of polymers with a non-silicone organic backbone (main chain) grafted with monomers containing a polysiloxane include the products sold under the names KP545, KP574 and KP575 by Shin-Etsu. One embodiment of the present disclosure may make use of a copolymer that may be obtained by radical polymerization starting with the following monomer mixture:

a) 60% by weight of tert-butyl acrylate;

b) 20% by weight of acrylic acid;

c) 20% by weight of a silicone macromer of formula:

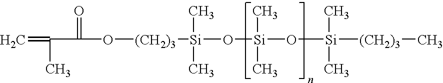

wherein n is an integer ranging from 5 to 700;

the weight percentages being calculated relative to the total weight of the monomers.

Another embodiment of the present disclosure may make use of a copolymer that may be obtained by radical polymerization starting with the following monomer mixture:

a) 80% by weight of tert-butyl acrylate;

b) 20% by weight of a silicone macromer of formula:

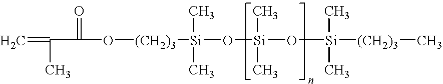

wherein n is an integer ranging from 5 to 700;

the weight percentages being calculated relative to the total weight of the monomers.

Another group of grafted silicone polymers with a non-silicone organic backbone (main chain) that may be used in compositions according to the present disclosure comprises grafted silicone copolymers which may be obtained by reactive extrusion-molding of a polysiloxane macromer with a reactive terminal function on a polymer of the polyolefin type comprising reactive groups capable of reacting with the terminal function of the polysiloxane macromer to form a covalent bond for grafting the silicone onto the main chain of the polyolefin. Non-limiting examples of such polymers are described, along with a process for their preparation, in PCT publication WO 95/00578.

Reactive polyolefins which may be used in the preparation of the above-described grafted silicone polymers may be chosen from, but are not limited to, polyethylenes and polymers of ethylene-derived monomers, for example propylene, styrene, alkylstyrene, butylene, butadiene, (meth)acrylates, vinyl esters, and equivalents, comprising reactive functions capable of reacting with the terminal function of the polysiloxane macromer. For example, the reactive polyolefins may be chosen from copolymers of ethylene or of ethylene derivatives and of monomers chosen from those comprising a carboxylic function, for example (meth)acrylic acid; those comprising an acid anhydride function, for example maleic anhydride; those comprising an acid chloride function, for example (meth)acryloyl chloride; those comprising an ester function, for example (meth)acrylic acid esters; and those comprising an isocyanate function. Polysiloxane macromers which may be used in the preparation of the above-described grafted silicone polymers may be chosen from polysiloxanes comprising a functionalized group, for example located at the end of the polysiloxane chain or close to the end of the said chain, chosen from alcohol, thiol, and epoxy groups and primary and secondary amines. For example, the polysiloxane macromers may be chosen from compounds of formula (VIII):

T-(CH₂)ₛ—Si—[—(OSiR⁵R⁶)ₜ—R⁷]_{y} (VII)

wherein T is chosen —NH₂, —NHR′, epoxy, —OH, and —SH groups; R⁵, R⁶, R⁷ and R′ are, independently of each other, chosen from C₁-C₆ alkyl, phenyl, benzyl, and C₆-C₁₂ alkylphenyl radicals and hydrogen atoms; s is an integer ranging from 2 to 100; t is an integer ranging from 0 to 1000 and y is an integer ranging from 1 to 3. Compounds of formula (VIII) may have a number-average molecular weight ranging from 5,000 to 300,000 g/mol, for example from 8,000 to 200,000 g/mol, for example from 9,000 to 40,000 g/mol.

In at least one embodiment of the present disclosure, the grafted silicone polymers having a polysiloxane backbone (main chain) grafted with non-silicone organic monomers, comprise a silicone main chain (or polysiloxane (≡Si—O)ₙ) onto which is grafted, within the said chain and also optionally on at least one of its ends, at least one organic group not comprising silicone.

In at least one embodiment of the present disclosure, a grafted silicone polymer having a polysiloxane backbone (main chain) grafted with non-silicone organic monomers may be used that comprises the result of a radical copolymerization between (1) at least one ethylenically unsaturated non-silicone anionic organic monomer and/or at least one ethylenically unsaturated non-silicone hydrophobic organic monomer, and (2) a silicone comprising within its chain at least one, for example several, functional group(s), for example thio functional groups, capable of reacting with the said ethylenic unsaturations of the said non-silicone monomers, forming a covalent bond.

In one aspect of the present disclosure, the said ethylenically unsaturated anionic monomers may be chosen, alone or as mixtures, from linear and branched unsaturated carboxylic acids, which may be partially or totally neutralized in the form of a salt. For example, the said ethylenically unsaturated anionic monomers may be chosen from acrylic acid, methacrylic acid, maleic acid, itaconic acid, fumaric acid, crotonic acid, and mixtures thereof. The said salts may be chosen from, for example, alkali metal salts, alkaline-earth metal salts, and ammonium salts. In one embodiment, in the final grafted silicone polymer, the organic group of anionic nature which comprises the result of the radical (homo)polymerization of at least one anionic monomer of unsaturated carboxylic acid type can, after reaction, be post-neutralized with a base (for example, sodium hydroxide or aqueous ammonia) in order to place it in the form of a salt.

In one embodiment, the said ethylenically unsaturated hydrophobic monomers may be chosen, alone or as a mixture, from acrylic acid esters of alkanols and/or methacrylic acid esters of alkanols. In one embodiment, the alkanols may be chosen from C₁-C₁₈ alkanols, for example, from C₁-C₁₂ alkanols. For example, the said ethylenically unsaturated hydrophobic monomers may be chosen from isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, stearyl (meth)acrylate, and mixtures thereof.

One group of grafted silicone polymers having a polysiloxane backbone (main chain) grafted with non-silicone organic monomers which may be used in compositions according to the present disclosure includes grafted silicone polymers comprising within their structure units of formula (IX″) and units of structure (IX) and/or (IX′) below:

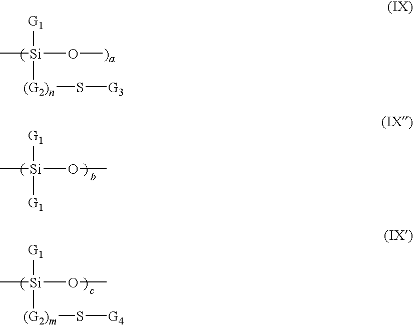

wherein the radicals G₁, which may be identical or different, are chosen from hydrogen atoms, C₁-C₁₀ alkyl radicals, and phenyl radicals; the radicals G₂, which may be identical or different, are chosen from C₁-C₁₀ alkylene radicals; G₃ is chosen from polymeric residues resulting from the (homo)polymerization of at least one anionic monomer containing ethylenic unsaturation; G₄ is chosen from polymeric residues resulting from the (homo)polymerization of at least one hydrophobic monomer containing ethylenic unsaturation; m and n are chosen from 0 and 1; a is an integer ranging from 0 to 50; b is an integer ranging from 10 to 350, c is an integer ranging from 0 to 50; with the proviso that at least one of the parameters a and c is not 0.

In one aspect of the present disclosure, the unit of formula (IX) has at least one, or even all, of the following characteristics:

the radicals G₁ are chosen from alkyl radicals, for example methyl radicals;

n is not zero, and the radicals G₂ are chosen from divalent C₁-C₃ radicals, for example propylene radicals;

G₃ is chosen from polymeric residues resulting from the (homo)polymerization of at least one monomer of the ethylenically unsaturated carboxylic acid type, for example acrylic acid and/or methacrylic acid;

G₄ is chosen from polymeric residues resulting from the (homo)polymerization of at least one monomer of the C₁-C₁₀ alkyl (meth)acrylate type, for example isobutyl and/or methyl (meth)acrylate.

Non-limiting examples of grafted silicone polymers of formula (VI) include polydimethylsiloxanes (PDMSs) onto which are grafted, via a thiopropylene-type secondary bond, mixed polymer units of the poly(meth)acrylic acid type and of the polyalkyl (meth)acrylate type. For example, as compounds corresponding to this definition, non-limiting mention may be made of poly (dimethyl/methyl siloxane) comprising 3-thiopropyl methyl acrylate/methyl methacrylate/methacrylic acid groups and Polysilicone-8 sold under the name VS80 by the company 3M.

Other non-limiting examples of grafted silicone polymers of formula (VI) include polydimethylsiloxanes (PDMSs) onto which are grafted, via a thiopropylene-type secondary bond, polymer units of the polyisobutyl (meth)acrylate type.

Grafted silicone polymers with a polysiloxane backbone grafted with non-silicone organic monomers which may be used in compositions according to the present disclosure may have a number-average molecular mass ranging from 10,000 to 1,000,000 g/mol, for example ranging from 10,000 to 100,000 g/mol.

In one embodiment, the grafted silicone polymers which may be used in compositions according to the present disclosure are chosen from copolymers of polydimethylsiloxane-grafted alkyl methacrylates, copolymers of isobutyl methacrylate, of acrylic acid and of a silicone macromer, and poly dimethyl/methyl siloxanes comprising 3-thiopropyl methyl acrylate/methyl methacrylate/methacrylic acid groups.

Compositions according to the present disclosure may further comprise at least one crosslinked silicone, for example a crosslinked elastomeric organopolysiloxane, for example a high molecular weight silicone compound of three-dimensional structure, having the viscoelastic properties of a supple solid material. Such an elastomer may be formed from high molecular weight polymer chains whose mobility is limited by a uniform network of crosslinking points. Such compounds may have the property of absorbing certain solvents, for example silicone solvents, and thus of thickening them, while at the same time giving the composition good cosmetic qualities, for example in terms of spreading.

Crosslinked organopolysiloxanes which may be used in compositions according to the present disclosure may be in the form of a dry powder, or in swollen form, in a solvent. The resulting product may be a gel or may be in dispersed form in an aqueous solution.

The synthesis of crosslinked organopolysiloxanes, such as those which may be used in compositions according to the present disclosure, is described in at least the following documents:

U.S. Pat. No. 5,266,321 to Kobayashi Kose,

U.S. Pat. No. 4,742,142 to Toray Silicone,

U.S. Pat. No. 5,654,362 to Dow Corning Corp.,

patent application FR 2 864 784.

The elastomeric organopolysiloxanes which may be used in compositions according to the present disclosure may be partially or totally crosslinked. In at least one aspect, the elastomeric organopolysiloxanes may be in the form of particles, which may have a number-average size ranging from 0.1 to 500 μm, for example from 3 to 200 μm, for example from 3 to 50 μm. These particles may have any shape and may be, for example, spherical, flat, or amorphous.

The elastomeric crosslinked organopolysiloxane which may be used in compositions according to the present disclosure may be obtained via crosslinking addition reactions of diorganopolysiloxanes comprising at least one hydrogen atom bonded to a silicon atom and of diorganopolysiloxanes comprising ethylenically unsaturated groups bonded to a silicon atom, for example in the presence of a platinum catalyst; or via dehydrogenation crosslinking coupling reactions between diorganopolysiloxanes bearing hydroxyl end groups and diorganopolysiloxanes containing at least one hydrogen atom bonded to a silicon atom, for example in the presence of an organotin compound; or via crosslinking coupling reactions of diorganopolysiloxanes bearing hydroxyl end groups and of hydrolysable organopolysilanes; or via thermal crosslinking of organopolysiloxanes, for example in the presence of an organoperoxide catalyst; or via crosslinking of organopolysiloxanes with high-energy radiation, for example with gamma rays, ultraviolet rays, and/or an electron beam.

In one embodiment, elastomeric crosslinked organopolysiloxanes which may be used in compositions according to the present disclosure are obtained via crosslinking addition reactions of diorganopolysiloxanes (X) comprising at least one hydrogen atom bonded to a silicon atom, and of diorganopolysiloxanes (XI) comprising at least two ethylenically unsaturated groups each bonded to a different silicon atom, for example in the presence of a platinum catalyst (XII), as described, for example, in patent application EP-A-295 886.

Compound (X) is, for example, an organopolysiloxane comprising at least two hydrogen atoms bonded to different silicon atoms in each molecule. Compound (X) may have any molecular architecture, for example, it may be in the form of a linear chain, a branched chain, or a cyclic structure. Compound (X) may have a viscosity at 25° C. ranging from 1 to 50,000 cSt, for example in order to have good miscibility with compound (XI).

The organic groups bonded to silicon atoms of compound (X) may be chosen from alkyl radicals, for example methyl, ethyl, propyl, butyl, and octyl radicals; substituted alkyl radicals, for example 2-phenylethyl, 2-phenylpropyl, and 3,3,3-trifluoropropyl radicals; aryl radicals, for example phenyl, tolyl, and xylyl; substituted aryl radicals, for example phenylethyl radicals; and substituted monovalent hydrocarbon-based groups such as epoxy groups, carboxylate ester groups, and mercapto groups. For example, Compound (X) may be chosen from methylhydrogenopolysiloxanes comprising trimethylsiloxy end groups, dimethylsiloxane-methylhydrogenosiloxane copolymers comprising trimethylsiloxy end groups, and dimethylsiloxane-methylhydrogenosiloxane cyclic copolymers.

Compound (XI) is, for example, a diorganopolysiloxane comprising at least two lower alkenyl groups (for example, C₂-C₄ alkenyl groups); the lower alkenyl group may be chosen, for example, from vinyl, allyl, and propenyl radicals. These lower alkenyl groups may be located in any position of the organopolysiloxane molecule; in at least one embodiment, they may be located at the chain ends of the organopolysiloxane molecule.

Compound (XI) may have a branched-chain, linear-chain, cyclic, or network structure. In at least one embodiment, Compound (XI) has a linear-chain structure. Compound (XI) may have a viscosity ranging from the liquid state to the gum state. In at least one embodiment, compound (XI) has a viscosity of at least 100 cSt at 25° C. In addition to the above-mentioned alkenyl groups, non-limiting examples of organic groups bonded to the silicon atoms in compound (XI) include alkyl radicals, for example methyl, ethyl, propyl, butyl, and octyl radicals; substituted alkyl radicals, for example 2-phenylethyl, 2-phenylpropyl, and 3,3,3-trifluoropropyl radicals; aryl radicals, for example phenyl, tolyl, and xylyl radicals; substituted aryl radicals, for example phenylethyl radicals; and substituted monovalent hydrocarbon-based groups, for example epoxy groups, carboxylate ester groups, and mercapto groups.

Compound (XI) may be chosen from, for example, but not limited to, methylvinylpolysiloxanes, methylvinylsiloxane-dimethylsiloxane copolymers, dimethylpolysiloxanes comprising dimethylvinylsiloxy end groups, dimethylsiloxane-methylphenylsiloxane copolymers comprising dimethylvinylsiloxy end groups, dimethyl-siloxane-diphenylsiloxane-methylvinylsiloxane copolymers comprising dimethylvinylsiloxy end groups, dimethylsiloxane-methylvinylsiloxane copolymers comprising trimethylsiloxy end groups, dimethylsiloxane-methylphenylsiloxane-methylvinylsiloxane copolymers comprising trimethylsiloxy end groups, methyl(3,3,3-trifluoropropyl)polysiloxane comprising dimethylvinylsiloxy end groups, and dimethylsiloxane-methyl(3,3,3-trifluoropropyl)siloxane copolymers comprising dimethylvinylsiloxy end groups. In one embodiment, compound (XI) may be obtained via reaction of dimethylpolysiloxanes comprising dimethylvinylsiloxy end groups and of methylhydrogenopolysiloxanes comprising trimethylsiloxy end groups, for example in the presence of a platinum catalyst.

In at least one embodiment, the sum of the number of ethylenic groups per molecule of compound (XI) and of the number of hydrogen atoms bonded to silicon atoms per molecule of compound (X) is at least 5.

In at least one embodiment, compound (X) may be added in an amount such that the molecular ratio between the total amount of hydrogen atoms bonded to silicon atoms in compound (X) and the total amount of all the ethylenically unsaturated groups in compound (XI) ranges from 1.5:1 to 20:1.

Compound (XII) is the catalyst for the crosslinking reaction and may be chosen from, for example, but not limited to, chloroplatinic acid, chloroplatinic acid-olefin complexes, chloroplatinic acid-alkenylsiloxane complexes, chloroplatinic acid-diketone complexes, platinum black, and platinum on a support. Compound (XII) may be added in a proportion ranging from 0.1 to 1000 parts by weight, for example from 1 to 100 parts by weight, as clean platinum metal per 1000 parts by weight of the total amount of compounds (X) and (XI).

The crosslinked organopolysiloxanes obtained via crosslinking addition reactions of compounds (X) and of compounds (XI) in the presence of compound (XII) may be a non-emulsifying compound or an emulsifying compound. As used herein, the term “non-emulsifying crosslinked organopolysiloxanes” is understood to mean crosslinked organopolysiloxanes not comprising any polyoxyalkylene units. As used herein, the term “emulsifying crosslinked organopolysiloxanes” is understood to mean crosslinked organopolysiloxane compounds comprising at least one polyoxyalkylene unit, for example polyoxyalkylene and/or polyoxypropylene.

The crosslinked organopolysiloxane particles may be conveyed in the form of a gel constituted by a crosslinked organopolysiloxane included in at least one hydrocarbon-based oil and/or one silicone oil. In such gels, the organopolysiloxane particles may be non-spherical. The crosslinked organopolysiloxane particles may also be in powder form, for example in the form of spherical powder.

Non-emulsifying crosslinked organopolysiloxanes such as those which may be used in compositions according to the present disclosure are described, as non-limiting examples, in U.S. Pat. Nos. 4,970,252, 4,987,169, 5,412,004, 5,654,362, and 5,760,116 and in patent application JP-A-601-194 009.

Non-limiting examples of non-emulsifying crosslinked organopolysiloxanes which may be used in compositions according to the present disclosure include those sold under the names KSG-6, KSG-15, KSG-16, KSG-18, KSG-31, KSG-32, KSG-33, KSG-41, KSG-42, KSG-43, KSG-44, and USG-103 by the company Shin-Etsu, DC9040, DC9041, DC9509, DC9505, DC9506, and DC9045 by the company Dow Corning, Gransil by the company Grant Industries, and SFE 839 by the company General Electric.

In at least one embodiment, the emulsifying crosslinked organopolysiloxanes which may be used in compositions according to the present disclosure comprise polyoxyalkylene-modified organopolysiloxanes formed from divinyl compounds, for example polysiloxanes comprising at least two vinyl groups, which can react with Si—H bonds of a polysiloxane. Emulsifying crosslinked organopolysiloxanes such as those which may be used in compositions according to the present disclosure are described, as non-limiting examples, in U.S. Pat. Nos. 5,236,986; 5,412,004; 5,837,793; and 5,811,487.

Non-limiting examples of emulsifying crosslinked organopolysiloxanes that may be used in compositions according to the present disclosure include those sold under the names KSG-21, KSG-20, KSG-30, and X-226146 by the company Shin-Etsu, and DC9010 and DC9011 by the company Dow Corning.

In one embodiment, the elastomeric crosslinked organopolysiloxane particles may be in the form of a powder of elastomeric crosslinked organopolysiloxane coated with silicone resin, for example with silsesquioxane resin, as described, as a non-limiting example, in U.S. Pat. No. 5,538,793.

Non-limiting examples of such elastomers include the products sold under the names KSP-100, KSP-101, KSP-102, KSP-103, KSP-104, and KSP-105 by the company Shin-Etsu.

Other non-limiting examples of elastomeric crosslinked organopolysiloxanes in powder form include hybrid silicone powders functionalized with fluoroalkyl groups, for example the product sold under the name KSP-200 by the company Shin-Etsu; and hybrid silicone powders functionalized with phenyl groups, for example the product sold under the name KSP-300 by the company Shin-Etsu.

In one embodiment, the crosslinked organopolysiloxanes may be in the form of dispersions of powders in water in the presence or absence of an emulsifier, for example the products sold under the names BY29-119, DC2-1997, EPSX001B, EPSX002B, and EPSX004A by Dow Corning.

In one embodiment, compositions according to the present disclosure may comprise at least one polysiloxane whose viscosity is greater than 100 cSt and that is present in the composition in an amount ranging from 0.1% to 30% by weight, for example from 0.1% to 20% by weight, for example from 0.1% to 10% by weight, relative to the total weight of the composition.

Compositions according to the present disclosure may further comprise at least one non-silicone polymer that may improve either the intrinsic properties of the composition, or the coating obtained during application to the hair, or both.

Such a polymer may be chosen from:

polymers that are soluble in an organic liquid medium, for example liposoluble polymers,

polymers that are dispersible in an organic solvent medium, for example polymers in the form of non-aqueous dispersions of polymer particles with a primary size of less than 1 μm, for example dispersions in silicone oils or hydrocarbon-based oils;

polymers in the form of aqueous dispersions of polymer particles with a primary size of less than 1 μm, which may be referred to as “lattices”; in this embodiment, the composition comprises an aqueous phase; and

water-soluble polymers; in this embodiment, the composition comprises an aqueous phase or the polymer is applied as a pre-treatment or post-treatment to the composition according to the present disclosure.

The at least one non-silicone polymer that may be used in the composition may be anionic, cationic, nonionic, or amphoteric.

Compositions according to the present disclosure may further comprise fillers, for example, but not limited to, substantially uncolored compounds that are solid at room temperature and atmospheric pressure, and insoluble in the composition, even when these ingredients are brought to a temperature above room temperature.

Fillers which may be used in compositions according to the present disclosure may be mineral or organic. These fillers may be particles of any shape, for example platelet-shaped, spherical, or oblong, independently of their crystallographic form (which may be, for example, lamellar, cubic, hexagonal, or orthorhombic). The filler particles may be solid, hollow, or porous, and they may be coated or uncoated.

Non-limiting examples of fillers that may be used in the compositions according to the present disclosure include mineral fillers, for example talc, natural and synthetic mica; kaolin; boron nitride, titanium dioxide, precipitated calcium carbonate; magnesium carbonate; magnesium hydrogen carbonate; hydroxyapatite; cerium oxide, and zirconium oxide.

In one aspect of the present disclosure, the fillers that may be used in the compositions according to the present disclosure may be chosen from mineral particles having a number-average primary size ranging from 0.1 to 30 μm, for example from 0.2 to 20 μm, for example from 0.5 to 15 μm. As used herein, the term “primary particle size” is understood to mean the maximum dimension that it is possible to measure between two diametrically opposite points on an individual particle. The size of the organic particles may be determined, for example, by transmission electron microscopy, by measuring the specific surface area via the BET method, or via laser granulometry.

In at least one embodiment, the mineral fillers that may be used in the compositions according to the present disclosure are chosen from talc, boron nitride, and titanium dioxide.

Further non-limiting examples of fillers that may be used in the compositions according to the present disclosure include organic fillers. As used herein, the term “organic filler” is understood to mean a polymer particle that may be derived from the polymerization of one or more monomers. The polymers constituting these organic particles may be crosslinked or non-crosslinked. The monomers used may, for example, be esters of methacrylic or acrylic acid, for example methyl acrylate and methacrylate, vinylidene chloride, acrylonitrile, and styrene, and derivatives thereof.

In one aspect of the present disclosure, the fillers that may be used in the compositions according to the present disclosure may be chosen from organic fillers having a number-average primary size ranging from 1 to 30 μm, for example from 1 to 20 μm, for example from 1 to 15 μm.

Non-limiting examples of organic fillers that may be used in cosmetic compositions according to the present disclosure include polyamide powders, acrylic polymer powders, for example powders of polymethyl methacrylate, acrylic copolymer powders, for example powders of polymethyl methacrylate/ethylene glycol dimethacrylate, of polyallyl methacrylate/ethylene glycol dimethacrylate, of ethylene glycol dimethacrylate/lauryl methacrylate copolymer, or of polyacrylate/alkyl acrylate, polystyrene powders, and polyethylene powders, for example powders of polyethylene/acrylic acid.

As organic fillers which may be used in cosmetic compositions according to the present disclosure, non-limiting mention may be made of:

polyamide (Nylon®) powders, for example those sold under the names Orgasol® 4000 and Orgasol® 2002 UD Nat Cos 204 by the company Atochem,

acrylic polymer powders, for example powders of polymethyl methacrylate, for example those sold under the name Covabead® LH85 and Covabead® PMMA by the company LCW and those sold under the name Micropearl® MHB sold by the company Matsumoto,

acrylic copolymer powders, for example powders of polymethyl methacrylate/ethylene glycol dimethacrylate, for example those sold under the name Dow Corning 5640 Microsponge® Skin Oil Adsorber by the company Dow Corning, and those sold under the name Ganzpearl® GMP-0820 by the company Ganz Chemical, of polyallyl methacrylate/ethylene glycol dimethacrylate, for example those sold under the name Polypore® L200 or Polypore® E200 sold by the company Amcol, of ethylene glycol dimethacrylate/lauryl methacrylate copolymer, for example those sold under the name Polytrap® 6603 by the company Dow Corning, and of polyacrylate/ethylhexyl acrylate, for example those sold under the name Techpolymer® ACX 806C by the company Sekisui,

polystyrene/divinylbenzene powders, for example those sold under the name Techpolymer® SBX8 by the company Sekisui,

polyethylene powders, for example powders of polyethylene/acrylic acid sold under the name Flobeads® by the company Sumitomo,

acrylic polymer microspheres, for example those made of the crosslinked acrylate copolymer Polytrap 6603 Adsorber® from the company RP Scherrer,

polyurethane powders, for example the powder of the copolymer of hexamethylene diisocyanate and of trimethylol hexyl lactone sold under the name Plastic Powder D-400® by the company Toshiki,

microcapsules of methyl acrylate and/or methacrylate polymers or copolymers, and microcapsules of copolymers of vinylidene chloride and of acrylonitrile, for example Expancel® from the company Expancel,

elastomeric crosslinked organopolysiloxane powders, for example those sold under the name Trefil Powder E-506C by the company Dow Corning, and

polyfluoro powders, for example powders of polytetrafluoroethylene, for example the product sold under the name MP 1400 by the company Dupont de Nemours.

In one embodiment, the organic fillers used in compositions according to the present disclosure are chosen from polyamide powders and polymethyl methacrylate powders.

Compositions according to the present disclosure may further comprise at least one oil thickener chosen from polymeric thickeners and mineral thickeners, and mixtures thereof.

The polymeric thickener may, for example, be an amorphous polymer formed by polymerization of an olefin. The olefin may, for example, be an elastomeric ethylenically unsaturated monomer.

Non-limiting examples of olefins include ethylenic carbide monomers, for example those comprising one or two ethylenic unsaturations and comprising from 2 to 5 carbon atoms, for example ethylene, propylene, butadiene, and isoprene.

The polymeric thickener may be capable of thickening or gelling the organic phase of the composition. As used herein, the term “amorphous polymer” is understood to mean a polymer that does not have a crystalline form. The polymeric thickener may also be a film-forming polymer.

In one embodiment, the polymeric thickener may be chosen from diblock, triblock, multiblock, radial, and star copolymers, and mixtures thereof.

Non-limiting examples of polymeric thickeners include those described in U.S. Patent Application Publication 2002/005562 and in U.S. Pat. No. 5,221,534.

In at least one embodiment, the polymeric thickener is chosen from amorphous block copolymers of styrene and of olefin.

In at least one embodiment, the polymeric thickener may be hydrogenated to reduce the residual ethylenic unsaturations after polymerization of the monomers.

For example, in one embodiment, the polymeric thickener is an optionally hydrogenated copolymer, comprising styrene blocks and ethylene/C₃-C₄ alkylene blocks.

Non-limiting examples of diblock copolymers, which may be hydrogenated, include styrene-ethylene/propylene copolymers and styrene-ethylene/butadiene copolymers, for example those sold under the name Kraton® G1701E by the company Kraton Polymers.

Non-limiting examples of triblock copolymers, which may be hydrogenated, include styrene-ethylene/propylene-styrene copolymers, styrene-ethylene/butadiene-styrene copolymers, styrene-isoprene-styrene copolymers and styrene-butadiene-styrene copolymers, for example those sold under the names Kraton® G1650, Kraton® G1652, Kraton® D1101, Kraton® D1102, and Kraton® D1160 by the company Kraton Polymers.

In at least one embodiment, a mixture of hydrogenated styrene-butadiene/ethylene-styrene triblock copolymers and of hydrogenated ethylene-propylene-styrene star polymers may be used; such a mixture may, for example, be in isododecane. Non-limiting examples of such mixtures include the products sold by the company Penreco under the trade names Versagel® M5960 and Versagel® M5670.

In at least one embodiment, a diblock copolymer such as those described above, for example a styrene-ethylene/propylene diblock copolymer, may be used as polymeric thickener.

A polymeric thickener may be present in compositions according to the present disclosure in an amount ranging from 0.1% to 10% by weight, for example ranging from 0.5% to 8% by weight, for example ranging from 1% to 5% by weight, relative to the total weight of the composition.

Compositions according to the present disclosure may further comprise at least one mineral oil-thickening agent, for example chosen from organophilic clays and fumed silicas.

As used herein, the term “organophilic clays” is understood to mean clays modified with chemical compounds that make the clay able to swell in oily media.

Clays are products that are already well known per se, and which are described, as non-limiting examples, in the publication “Minéralogie des argues [Mineralogy of clays]” by S. Caillère, S. Hénin, M. Rautureau, 2nd Edition 1982, Masson, the disclosure of which is included herein by way of reference.

Clays are silicates containing a cation that may be chosen from calcium, magnesium, aluminum, sodium, potassium, and lithium cations, and mixtures thereof.

Non-limiting examples of clays include clays of the smectite family such as montmorillonites, hectorites, bentonites, beidellites, and saponites, and also clays of the vermiculite, stevensite, and chlorite families.

Clays which may be used in compositions according to the present disclosure may be of natural or synthetic origin. In at least one embodiment, the clays are cosmetically compatible and acceptable with keratin materials such as the skin and the hair.

Non-limiting examples of organophilic clays which may be used in compositions according to the present disclosure include montmorillonite, bentonite, hectorite, attapulgite, and sepiolite, and mixtures thereof. In at least one embodiment, the clay is a bentonite, a hectorite, or a mixture thereof.

The organophilic clays that may be used in compositions according to the present disclosure may be modified with a chemical compound chosen from quaternary amines, tertiary amines, amine acetates, imidazolines, amine soaps, fatty sulfates, alkyl aryl sulfonates, and amine oxides, and mixtures thereof.

Non-limiting examples of organophilic clays that may be used in compositions according to the present disclosure include quaternium-18 bentonites, for example those sold under the names Bentone 3, Bentone 38, and Bentone 38V by the company Rheox, Tixogel VP by the company United Catalyst, and Claytone 34, Claytone 40, and Claytone XL by the company Southern Clay; stearalkonium bentonites, for example those sold under the names Bentone 27 by the company Rheox, Tixogel LG by the company United Catalyst, and Claytone AF and Claytone APA by the company Southern Clay; quaternium-18/benzalkonium bentonites, for example those sold under the names Claytone HT and Claytone PS by the company Southern Clay.

Fumed silicas may be obtained by high-temperature hydrolysis of a volatile silicon compound in an oxhydric flame, producing a finely divided silica. This process makes it possible to obtain hydrophilic silicas having a large number of silanol groups at their surface. Non-limiting examples of such silicas include those sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

It is possible to chemically modify the surface of a silica via a chemical reaction generating a reduction in the number of silanol groups. For example, it is possible to substitute silanol groups with hydrophobic groups: a hydrophobic silica is then obtained.

The above-mentioned hydrophobic groups may be chosen from:

trimethylsiloxyl groups, for example those obtained by treating fumed silica in the presence of hexamethyldisilazane. Silicas thus treated may be known as “silica silylate” according to the CTFA (6th Edition, 1995). They include the products sold, for example, under the references Aerosil R812® by the company Degussa and Cab-O-Sil TS-530® by the company Cabot; and

dimethylsilyloxyl and polydimethylsiloxane groups, for example those obtained by treating fumed silica in the presence of polydimethylsiloxane or dimethyldichlorosilane. Silicas thus treated may be known as “silica dimethyl silylate” according to the CTFA (6th Edition, 1995). They include the products sold, for example, under the references Aerosil R972® and Aerosil R974® by the company Degussa and Cab-O-Sil TS-610® and Cab-O-Sil TS-720® by the company Cabot.

Fumed silicas that may be used in compositions according to the present disclosure may have a particle size that may be nanometric to micrometric, for example ranging from about 5 to 200 nm.

In at least one embodiment, compositions according the present disclosure may comprise an organomodified bentonite or hectorite as a mineral thickener.

The mineral oil-thickening agent may be present in compositions according to the present disclosure in an amount ranging from 0.1% to 8% by weight, for example from 0.2% to 6% by weight, for example from 0.5% to 4% by weight, relative to the total weight of the composition.

Compositions according to the present disclosure may further comprise at least one agent chosen from, for example, reducing agents, fatty substances, plasticizers, softeners, antifoams, moisturizers, UV-screening agents, mineral colloids, peptizers, solubilizers, fragrances, proteins, vitamins, propellants, oxyethylenated and non-oxyethylenated waxes, paraffins, C₁₀-C₃₀ fatty acids such as stearic acid and lauric acid, and C₁₀-C₃₀ fatty amides such as lauric diethanolamide.

The above-mentioned additives may be present in an amount for each of them ranging from 0.01% to 20% by weight relative to the total weight of the composition.

A person skilled in the art will take care to select this or these optional additional agents such that the advantageous properties intrinsically associated with the formation of the coating in accordance with the present disclosure are not, or are not substantially, adversely affected.

Compositions according to the present disclosure may be in any form that is suitable for application to the hair, for example a cream, a mousse, a stick, a dispersion of vesicles, for example of ionic or nonionic lipids, a two-phase or multiphase lotion, an aerosol spray, for example lacquers, a powder, or a paste.

Compositions according to the present disclosure are anhydrous compositions. As used herein, the term “anhydrous composition” is understood to mean a composition containing less than 2% by weight of water, for example less than 0.3% water, for example substantially free of water, the water present resulting solely from the residual water provided by the mixed ingredients.

Compositions according to the present disclosure may be used on dry or wet hair. The optional additional agents described previously, when they are present, may be applied to the hair simultaneously with compositions according to the present disclosure or separately. Compositions according to the present disclosure may be rinsed out or left in; it is also possible subsequently to wash the hair, this washing not being required.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow are intended to illustrate the present disclosure without, however, being limiting in nature.

### EXAMPLES

### Examples

Compositions according to the present disclosure were prepared from the following compounds:

| | Ex 1 | Ex 2 | Ex 3 | Ex 4 | Ex 5 | Ex 6 |
|---|---|---|---|---|---|---|
| α,ω-Dihydroxylated polydi- | 20 g | 20 g | 20 g | 20 g | 20 g | — |
| methylsiloxane/cyclopenta- | | | | | | |
| dimethylsiloxane mixture (14.7/85.3) | | | | | | |
| sold under the name DC1501 Fluid (*) | | | | | | |
| Linear silicone DC200 Fluid 500 000 cSt (*) | — | — | — | — | — | 3 g |
| Bio-PSA ® 7-4400 (*) | 10 g | — | 10 g | 10 g | 10 g | 10 g |
| Bio-PSA ® 7-4500 (*) | — | 10 g | — | — | — | — |
| Isododecane | — | — | 30 g | — | — | — |
| Volatile linear silicone DC200 Fluid 1 cSt (*) | — | — | — | qs | — | — |
| | | | | 100 g | | |
| Ethanol | — | — | — | — | 20 g | — |
| Volatile cyclic silicone DC245 Fluid (*) | qs | qs | qs | — | qs | qs |
| | 100 g | 100 g | 100 g | | 100 g | 100 g |

(*) sold by Dow Corning

0.3 g of the composition was applied to a 1 g lock of clean, wet hair of tone depth 4. After a leave-on time of 15 minutes, the lock was dried with a hairdryer for 2 minutes. A lock whose strands were individualized and coated is obtained. This coating was shampoo-resistant.

| | Ex 7 | Ex 8 | Ex 9 | Ex 10 | Ex 11 | Ex 12 |
|---|---|---|---|---|---|---|
| α,ω-Dihydroxylated polydi- | 20 g | 20 g | 20 g | 20 g | 20 g | — |
| methylsiloxane/cyclopenta- | | | | | | |
| dimethylsiloxane mixture (14.7/85.3) | | | | | | |
| sold under the name DC1501 Fluid (*) | | | | | | |
| Linear silicone DC200 Fluid 500 000 cSt (*) | — | — | — | — | — | 3 g |
| Bio-PSA ® 7-4400 (*) | 10 g | — | 10 g | 10 g | 10 g | 10 g |
| Bio-PSA ® 7-4500 (*) | — | 10 g | — | — | — | — |
| Mica nacre coated with brown iron | 10 g | 10 g | 10 g | 10 g | 10 g | 10 g |
| oxide, sold by Eckart under the | | | | | | |
| name Prestige Bronze | | | | | | |
| Isododecane | — | — | 30 g | — | — | 30 g |
| Volatile linear silicone DC200 Fluid 1 cSt (*) | — | — | — | qs | — | — |
| | | | | 100 g | | |
| Ethanol | — | — | — | — | 20 g | — |
| Volatile cyclic silicone DC245 Fluid (*) | qs | qs | qs | — | qs | qs |
| | 100 g | 100 g | 100 g | | 100 g | 100 g |

(*) sold by Dow Corning

0.8 g of the composition was applied to a 1 g lock of clean, wet hair. After a leave-on time of 15 minutes, the lock was dried with a hairdryer for 2 minutes. A colored lock whose strands were individualized and whose color is shampoo-resistant was obtained.

### Examples with Organic and Mineral Pigments

| | Ex 13 | Ex 14 | Ex 15 | Ex 16 | Ex 17 | Ex 18 |
|---|---|---|---|---|---|---|
| α,ω-Dihydroxylated polydi- | 20 g | 20 g | 20 g | 20 g | — | — |
| methylsiloxane/cyclopenta- | | | | | | |
| dimethylsiloxane mixture (14.7/85.3) | | | | | | |
| sold under the name DC1501 Fluid (*) | | | | | | |
| Linear silicone DC200 Fluid 500 000 cSt (*) | — | — | — | — | 3 g | 3 g |
| Bio-PSA ® 7-4400 (*) | 10 g | 10 g | 10 g | 10 g | 10 g | 10 g |
| Red iron oxide sold by Sun under | 10 g | — | 10 g | — | 10 g | — |
| the name Sunpuro | | | | | | |
| Disperse Red 122, CI 73915 sold by | — | 10 g | — | 10 g | — | 10 g |
| Sun under the name Sunfast | | | | | | |
| Magenta 122 | | | | | | |
| Isododecane | — | — | 30 g | 30 g | 30 g | 30 g |
| Volatile cyclic silicone DC245 Fluid (*) | qs | qs | qs | qs | qs | qs |
| | 100 g | 100 g | 100 g | 100 g | 100 g | 100 g |

(*) sold by Dow Corning

0.8 g of the composition was applied to a 1 g lock of clean, wet hair. After a leave-on time of 15 minutes, the lock was dried with a hairdryer for 2 minutes. A colored lock whose strands were individualized and whose color is shampoo-resistant was obtained.

### Examples with Thickened Formulations

| | Ex 19 | Ex 20 | Ex 21 | Ex 22 | Ex 23 | Ex 24 |
|---|---|---|---|---|---|---|
| α,ω-Dihydroxylated polydi- | 20 g | 20 g | 20 g | 20 g | — | — |
| methylsiloxane/cyclopenta- | | | | | | |
| dimethylsiloxane mixture (14.7/85.3) | | | | | | |
| sold under the name DC1501 Fluid (*) | | | | | | |
| Linear silicone DC200 Fluid 500 000 cSt (*) | — | — | — | — | 3 g | 3 g |
| Bio-PSA ® 7-4400 (*) | 10 g | 10 g | 10 g | 10 g | 10 g | 10 g |
| Mica nacre coated with brown iron | — | 10 g | — | 10 g | — | 10 g |
| oxide, sold by Eckart under the name | | | | | | |
| Prestige Bronze | | | | | | |
| Isododecane | — | — | 30 g | 30 g | 30 g | 30 g |
| Smectite at 10% in isododecane, sold | 25 g | 25 g | 25 g | 25 g | 25 g | 25 g |
| by Elementis under the name Bentone | | | | | | |
| Gel ISD v | | | | | | |
| Volatile cyclic silicone DC245 Fluid (*) | qs | qs | qs | qs | qs | qs |
| | 100 g | 100 g | 100 g | 100 g | 100 g | 100 g |

(*) sold by Dow Corning

### For Examples 19, 21 and 23

0.3 g of the composition was applied to a 1 g lock of clean, wet hair of tone depth 4. After a leave-on time of 15 minutes, the lock was dried with a hairdryer for 2 minutes. A lock whose strands were individualized and coated was obtained. This coating was shampoo-resistant.

### For Examples 20, 22 and 24

0.8 g of the composition was applied to a 1 g lock of clean, wet hair. After a leave-on time of 15 minutes, the lock was dried with a hairdryer for 2 minutes. A colored lock whose strands were individualized and whose color was shampoo-resistant was obtained.

### Comparative Examples

The following compositions 25, 25bis, 26, and 26bis were prepared. Examples 25 and 26, according to the present disclosure, were prepared with an amount of Bio-PSA® higher than 1% whereas the comparative examples 25bis and 26bis were prepared with 1% of Bio-PSA®.

| | Ex 25 | Ex 25bis | Ex 26 | Ex 26bis |
|---|---|---|---|---|
| CYCLOPENTASILOXANE | 20 | 20 | 20 | 20 |
| (and) DIMETHICONOL sold | | | | |
| by DOW CORNING under | | | | |
| the name DC 1501 FLUID | | | | |
| Bio-PSA ® 7-4400 (*) | 10 | 1 | 10 | 1 |
| Mica nacre coated with brown | 10 | 10 | — | — |
| iron oxide, sold by Eckart | | | | |
| under the name Prestige | | | | |
| Bronze | | | | |
| Isododecane | — | — | 30 | 30 |
| Smectite at 10% in | 25 | 25 | 25 | 25 |
| isododecane, sold by | | | | |
| Elementis under the name | | | | |
| Bentone Gel ISD v | | | | |
| Volatile cyclic silicone | Qsp 100 | Qsp 100 | Qsp 100 | Qsp 100 |
| DC245 Fluid (*) | | | | |

(*) sold by Dow Corning

Each composition was applied on locks (1 g) having of tone depth of 4 (corresponding to a clear chestnut color). For the examples 25 and 25bis, the amount of the composition applied on the locks was 0.8 g. For the examples 26 and 26bis, the amount of the composition applied on the locks was 0.3 g.

The locks were then washed with a shampoo (ultra doux camomille from Garnier).

For the locks treated with the compositions 25 and 25bis, the color was evaluated in the L*a*b* system, before and after the shampoo step by using a spectrocolorimeter Minolta CM-3600d, illuminant D65.

According to this system, L* indicates the lightness. The lower the value of L*, the more intense the color of the hair. The chromaticity coordinates are expressed by the parameters a* and b*, a* indicating the axis of red/green shades and b* the axis of yellow/blue shades.

ΔE, which is the color variation between a colored lock and a colored lock after 1 shampoo, is obtained from the following formula:

ΔE=\sqrt{(L*−Lₒ*)²+(a*−aₒ*)²+(b*−bₒ*)²}

wherein L* indicates lightness and a* and b* are the chromaticity coordinates of the colored locks after 1 shampoo whereas L₀* indicates the lightness and a₀* and b₀* are the chromaticity of the colored locks before shampoo. The lower the value of ΔE, the more resistant to shampoo is the color of the hair.

The obtained results are reported in the table below:

| | | L* | a* | b* | ΔE |
|---|---|---|---|---|---|
| Ex 25 | Before shampoo | 41.99 | 12.27 | 18.83 | — |
| Ex 25 | After shampoo | 40.39 | 10.16 | 16.48 | 3.5 |
| Ex 25bis | Before shampoo | 38.26 | 11.11 | 17.33 | — |
| Ex 25bis | After shampoo | 21.9 | 4.31 | 5.9 | 21 |

These results show that when the Bio-PSA® is used in an amount higher than 1%, the resulting color exhibits a better resistance to shampoo.

For examples 26 and 26bis, the evaluation was conducted qualitatively. By touching the lock of example 26 after and before shampooing, it can be felt that after the shampoo a coating remains present on the hair and the hair has greater body. With the lock of example 26bis, after shampooing, no coating can be felt. The feel is very near that of the natural hair before treatment with composition 26 bis.

What is claimed is:

1. An anhydrous composition for the treatment of keratin fibers comprising:
at least one copolymer based on a silicone resin and a fluid silicone,
at least one volatile silicone chosen from linear and cyclic volatile silicones, and
at least one non-volatile linear polydimethylsiloxane with a viscosity of greater than 5 cSt,
wherein the at least one copolymer is present in the composition in an amount greater than 1% by weight relative to the total weight of the composition, and wherein the composition does not comprise coloring pigments.

2. The composition according to claim 1, wherein the silicone resin is present in the at least one copolymer in an amount ranging from 40% to 70%, the fluid silicone is present in the at least one copolymer in an amount ranging from 30% to 60%, and the sum of the percentages of silicone resin and of fluid silicone is equal to 100.

3. The composition according to claim 2, wherein the silicone resin is present in the at least one copolymer in an amount ranging from 55% to 65% and the fluid silicone is present in the at least one copolymer in an amount ranging from 35% to 45%.

4. The composition according to claim 1, wherein the at least one copolymer is present in the composition in an amount ranging from greater than 1% to 40% by weight, relative to the total weight of the composition.

5. The composition according to claim 4, wherein the at least one copolymer is present in the composition in an amount ranging from 1.5% to 20% by weight, relative to the total weight of the composition.

6. The composition according to claim 1, wherein the at least one volatile silicone is chosen from octamethylcyclotetrasiloxane, decamethylcyclopentasiloxane, dodecamethylcyclohexasiloxane, and mixtures thereof.

7. The composition according to claim 6, wherein the at least one volatile silicone is chosen from decamethylcyclopentasiloxane.

8. The composition according to claim 1, wherein the at least one non-volatile linear polydimethylsiloxane is chosen from polydimethylsiloxanes; alkyl dimethicones; polyphenylmethylsiloxanes, phenyl trimethicones and vinyl methyl methicones; silicones modified with groups chosen from optionally fluorinated aliphatic and aromatic groups and hydroxyl, thiol and amine groups.

9. The composition according to claim 1, wherein the at least one non-volatile linear polydimethylsiloxane has a viscosity ranging from 100 cSt to 4,000,000 cSt at 25° C.

10. The composition according to claim 1, further comprising at least one non-silicone solvent.

11. The composition according to claim 10, wherein the at least one non-silicone solvent is chosen from hydrocarbons and alcohols.

12. The composition according to claim 1, further comprising at least one thickener.

13. The composition according to claim 12, wherein the at least one thickener is an organomodified clay.

14. The composition according to claim 1, wherein the keratin fibers are human hair.

15. A method of treating keratin fibers, comprising the application of an anhydrous composition comprising:
at least one copolymer based on a silicone resin and a fluid silicone,
at least one volatile silicone chosen from linear and cyclic volatile silicones, and
at least one non-volatile linear polydimethylsiloxane with a viscosity of greater than 5 cSt,
wherein the at least one copolymer is present in the composition in an amount greater than 1% by weight relative to the total weight of the composition, and wherein the composition does not comprise coloring pigments.

16. The method according to claim 15, further comprising rinsing or washing the keratin fibers after a leave-in time.

17. The method according to claim 15, wherein the keratin fibers are human hair.

18. An anhydrous hair dyeing composition, comprising:
at least one copolymer based on a silicone resin and a fluid silicone,
at least one volatile silicone chosen from linear and cyclic volatile silicones,
at least one non-volatile linear polydimethylsiloxane with a viscosity of greater than 5 cSt at 25° C., and
at least one coloring pigment,
wherein the at least one copolymer is present in the composition in an amount greater than 1% by weight relative to the total weight of the composition, and wherein the at least one coloring pigment is present in the composition in a total amount greater than 5% relative to the total weight of the composition.

19. The composition according to claim 18, wherein the silicone resin is present in the at least one copolymer in an amount ranging from 40% to 70%, the fluid silicone is present in the at least one copolymer in an amount ranging from 30% to 60%, and the sum of the percentages of silicone resin and of fluid silicone is equal to 100.

20. The composition according to claim 19, wherein the silicone resin is present in the at least one copolymer in an amount ranging from 55% to 65% and the fluid silicone is present in the at least one copolymer in an amount ranging from 35% to 45%.

21. The composition according to claim 18, wherein the at least one copolymer is present in the composition in an amount ranging from greater than 1% to 40% by weight, relative to the total weight of the composition.

22. The composition according to claim 21, wherein the at least one copolymer is present in the composition in an amount ranging from 1.5% to 20% by weight, relative to the total weight of the composition.

23. The composition according to claim 18, wherein the at least one volatile silicone is chosen from octamethylcyclotetrasiloxane, decamethylcyclopentasiloxane, dodecamethylcyclohexasiloxane, and mixtures thereof.

24. The composition according to claim 23, wherein the at least one volatile silicone is chosen from decamethylcyclopentasiloxane.

25. The composition according to claim 18, wherein the at least one non-volatile linear polydimethylsiloxane is chosen from polydimethylsiloxanes; alkyl dimethicones; polyphenylmethylsiloxanes, phenyl trimethicones and vinyl methyl methicones; silicones modified with groups chosen from optionally fluorinated aliphatic and aromatic groups and hydroxyl, thiol and amine groups.

26. The composition according to claim 18, wherein the at least one non-volatile linear polydimethylsiloxane has a viscosity ranging from 100 cSt to 4,000,000 cSt at 25° C.

27. The composition according to claim 18, wherein the at least one coloring pigment is chosen from natural pigments and nacres.

28. The composition according to claim 18, wherein the at least one coloring pigment is present in the composition in a total amount that is not more than 40%, relative to the total weight of the composition.

29. The composition according to claim 28, wherein the at least one coloring pigment is present in the composition in a total amount that is not more than 20%, relative to the total weight of the composition.

30. The composition according to claim 18, further comprising at least one non-silicone solvent.

31. The composition according to claim 30, wherein the at least one non-silicone solvent is chosen from hydrocarbons and alcohols.

32. The composition according to claim 18, further comprising at least one thickener.

33. The composition according to claim 32, wherein the at least one thickener is an organomodified clay.

34. The composition according to claim 18, wherein the keratin fibers are human hair.

35. A method of treating keratin fibers, comprising the application of an anhydrous composition comprising:
at least one copolymer based on a silicone resin and a fluid silicone,
at least one volatile silicone chosen from linear and cyclic volatile silicones,
at least one non-volatile linear polydimethylsiloxane with a viscosity of greater than 5 cSt at 25° C., and
at least one coloring pigment,
wherein the at least one copolymer is present in the composition in an amount greater than 1% by weight relative to the total weight of the composition, and wherein the at least one coloring pigment is present in the composition in a total amount greater than 5% relative to the total weight of the composition.

36. The method according to claim 35, further comprising rinsing or washing the keratin fibers after a leave-in time.

37. The method according to claim 35, wherein the keratin fibers are human hair.
